(19) United States
(12) Patent Application Publication
(10) Pub. No.: US 2023/0166006 A1
(43) Pub. Date: Jun. 1, 2023

(54) METHODS AND COMPOSITIONS FOR PERIAPICAL TISSUE REGENERATION

(71) Applicant: Khalid Alhezaimi, Newport Beach, CA (US)

(72) Inventor: Khalid Alhezaimi, Newport Beach, CA (US)

(21) Appl. No.: 18/073,246

(22) Filed: Dec. 1, 2022

Related U.S. Application Data
(60) Provisional application No. 63/284,771, filed on Dec. 1, 2021.

(51) Int. Cl. A61L 27/54; A61L 27/24; A61L 27/26; A61L 31/04; A61L 24/10

(52) U.S. Cl. CPC A61L 27/54; A61L 27/24; A61L 27/26; A61L 31/041; A61L 24/102; A61L 2300/414; A61L 2300/406

(57) ABSTRACT

Provided are methods and compositions for inducing periapical tissue regeneration and repair.

### CROSS REFERENCE TO RELATED APPLICATIONS

This application claims priority under 35 U.S.C. § 119 to U.S. Provisional Application Ser. No. 63/284,771, filed Dec. 1, 2021, the disclosures of which are incorporated herein by reference.

### FIELD OF THE INVENTION

The invention generally concerns methods of tissue engineering, regeneration and repair, and more particularly relates to methods and compositions for treating periodontal, periapical and apical osseous and soft mineralized tissue.

### BACKGROUND

Periradicular/periapical/apical lesion around vital and non vital teeth and unresolved periapical lesions in previously root canal treated teeth or previously initiated root canal treatment or with history of trauma with persistent apical or lateral bony defect of periodontal or non odontogenic origin can be treated with periapical, or lateral surgery. Many studies have been conducted to assess the best approach to this procedure. The ability to stimulate regeneration of lost soft and hard tissues is an important criterion in the selection of treatment approach. Regeneration is restoration of damaged tissues by the same tissues that mimic the original architectural form and function. On the other hand, repair is restoration of damaged tissues by new tissues that differ in function and architecture from the original tissues, which include all or one of the following tissues: periodontal ligaments, bone and cementum.

Growth factors are proteins involved in the regulation of cellular events including during wound tissue repair and regeneration. Intracellular signaling pathways are induced after the growth factors bind to specific cell membrane receptors of the target cells. This results in the activation of genes that can eventually alter cellular activity and phenotype. Experimental studies have shown that growth factors can potentially enhance tissue regeneration via series of events including cell chemo-attraction, differentiation and proliferation. These biological mediators have been studied extensively.

Platelet-derived growth factor (PDGF) presents as dimers of A, B and C polypeptide chains. Five isomeric forms of PDGF have been identified: PDGF-AA, PDGF-AB, PDGF-BB, PDGF-CC and PDGF-DD.

PDGF receptor signalling plays a significant role in regulation of the proliferation and cells migration including osteoblasts and fibroblasts. For example, PDGF alpha-receptors (or A-type receptors) bind only to three isoforms with high affinity while beta-receptors (or B-type receptors) bind to PDGF-BB with high affinity. This may explain the variations in impact and effect of different PDGF isoforms and their function.

### SUMMARY

The disclosure provides a method of promoting periapical and apical periodontal tissue healing, repair and regeneration, the method comprising direct or indirect application an apical or periapical site with a composition consisting of recombinant Platelet Derived Growth Factor (rPDGF). In one embodiment, the rPDGF is recombinant human PDGF (rhPDGF). In another or further embodiment, the composition containing the rPDGF is selected from the group consisting of collagen, gelatin hydrogels, fibrin gels, heparin, reverse phase polymers, poloxamers, poly-lactic acid (PLA), poly-glycolic acid (PGA), co-polymers of PLA and PGA (PLGA), heparin-conjugated PLGA carriers, and inorganic materials. In still a further embodiment, the inorganic material is calcium phosphates and/or beta tricalcium phosphate (R-TCP). In another embodiment, the collagen materials are selected from Type I collagen, Type II collagen, Type III collagen, bovine collagen, human collagen, porcine collagen, equine collagen, avian collagen, and any combination thereof. In another embodiment, the method further comprises administering antibiotics, antifungals or a combination thereof. In another embodiment, the apical or periapical site is a site of injury. In another embodiment, the injury is the result of a periodontal infection or surgery such as a root canal.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-C shows radiographic and histological images of the experimental teeth (A) At 8 weeks, periapical radiolucencies associated with the roots of all pre-contaminated premolars can be seen. (B) Six months postnon-surgical endodontic treatment radiographic image shows none healed periapical lesions around second premolar (P2), third premolar (P3), and fourth premolar (P4) and normal periapical area related to the non-treated first premolar (P1). (C) Reconstructed micro-CT image. Bone loss in the periapical areas (arrows) was defined as region of interest (ROI).

FIG. 2A-F show representative sections from the apical curettage group and apicoectomy+mineral trioxide aggregate (MTA) group. (A) Light microscopy section showing first (P1) and second (P2) premolars. Four months after endodontic treatment, P1 was treated with apical curettage only and P2 with apicoectomy+MTA root-end filling. In the area of P1, apical bone, PDL loss, and absence of cementum regeneration are evident. In the area of P2, complete closure of the root apices with newly formed cementum, loss of apical PDL, and absence of radicular bone regeneration are seen. A fibrous-like capsulation is seen between the newly formed cementum (NFC) and the MTA. No direct contact between the NFC and MTA can be observed. (original magnification ×4). (B) A corresponding axial plane μCT section taken for P1 is shows evidence of apical root resorption, absence of lamina dura, and significant apical bone loss. (C) A corresponding micro-CT of the histological image of P2 shows evidence of hard tissue formation covering the apices. No direct contact with the root-end filling material can be noted. (D) Reconstructed axial plane μCT images taken for P2 and P3 show evidence of large apical bone loss communicating with the mandibular canal. (E) A sagittal plane of reconstructed μCT section of the apical 3 mm shows a significant widening of the PDL around P2 and P3 as compared to P1. (F) Axial piano of μCT section for P1 treated with curettage alone shows evidence of bone loss and initial communication with the mandibular canal.

FIG. 3A-G shows (A) μCT taken 16 months following apicoectomy+MTA root-end filling, showing P2 and P3. Partial resolution of the periapical radiolucencies, absence of apical lamina dura and incomplete alveolar bone reformation are evident. (B) Light microscopy section showing P2 that had MTA root-end filling. Incomplete apical bone regeneration, and absence of newly formed cementum on the root apices are evident (original magnification ×2). (C) Light microscopy section showing absence of newly formed cementum covering the MTA, absence of apical PDL, and presence of abundant inflammatory cells (original magnification ×4). (D) Higher magnification showing newly formed cementum (NFC— blue arrow) not covering the MTA. (RD—Radicular Dentin). (original magnification ×8). (E) Evidence of old cementum attachment (red arrow) to NFC (blue arrow) (original magnification ×8). (F) Thickness of the newly formed cementum is reduced significantly in proximity to the MTA. No direct contact with the MTA can observed (original magnification ×16). (G) At higher magnification using light and fluorescent microscopy, respectively. Both samples show gaps between the newly formed cementum layer and the MTA. The gaps appear to be filled with fibrous-like tissue and inflammatory cells (original magnification ×32).

FIG. 4A-I show representative light microscopy (A-F) and reconstructed virtual μCT sections (G-I) taken from the apicoectomy+MTA+rhPDGF and apical curettage+rhPDGF groups. (A) Section showing complete regeneration of the apical area with presence of newly formed cementum, PDL, and bone (original magnification ×4). (B-C) Higher magnifications (×8 and ×16, respectively) of the same specimens showing complete regeneration of cementum, PDL and apical bone without evidence of ankyloses. (D) Higher magnification of the same specimens showing lack of direct contact between the MTA and the newly formed cementum and presence of fibrous-like tissue air the gaps between them. (original magnification ×24). (E-F) Light and fluorescent light microscopy showing the characteristics of the fibrous-like tissue (blue arrow). It does not resemble the shape, form, insertion and orientation of collagen fibres (original magnification ×32). (G-H) μCT of maxillary premolars (P1 and P2), 2 years following apicoectomy+MTA 4 rhPDGF therapeutic approaches showing presence of normal lamina dura and complete resolution of periapical lesions. (I) P1 (MTA+rhPDGF) and P2 (curettage+rhPDGF) shows complete resolution of chronic apical periodontitis and both are in close proximity to the maxillary sinus (MS). No loss of lamina dura around P1 or P2 can be noted and both have similar width compared to pristine lamina dura space around the canine control group.

FIG. 5A-E shows representative reconstructed virtual μCT (A-C) and light: microscopy sections (D-E) taken from the apical curettage and PDGF group. (A) Micro-CT section showing normal lamina dura in the periapical area of P1 and complete resolution of the periapical radiolucency. No evidence of bone resorption is noted despite the proximity to the maxillary sinus. (B) Serial sagittal section of the canine and adjacent experimental tooth P1). No difference between the width of the lamina dura of the vital tooth and the treated one is noted when apical curettage followed by application of PDGF approach was used. (C) H&E stained section showing P2 and P3 with complete regeneration of cementum, PDL and apical bone. (original magnification ×4). (D) Higher magnification of the same specimen for P1 showing complete closure of the apex with a thick layer of newly formed cementum, PDL and bone. (original magnification ×3).

### DETAILED DESCRIPTION

As used herein and in the appended claims, the singular forms “a,” “an,” and “the” include plural referents unless the context clearly dictates otherwise. Thus, for example, reference to “a protein” includes a plurality of such proteins and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Reports of complete bony periradicular or radicular lesion resolution following apical surgery (AS) vary in the literature. Variations are due to different methodologies, absence of histological and 3D imaging evidance and data analyses among the studies. The disclosure examines periradicular, lateral and radicular regeneration and repair and is the first to report long-term histological and μCT 3D imaging of healing patterns following AS in vivo. The disclosure demonstrates that curettage alone as therapeutic technique resulted in partial repair of the involved tissues without regeneration. These finding agree with previous reports indicating that apical curettage did not yield resolution of chronic apical lesions and did not stimulate tissues regeneration. This is most probably due to microorganism colonization and presence of pathological/necrotic soft tissue in the apical, lateral, periradicular and radicular tissues,

The disclosure provides compositions and methods for treating damaged tissue. More particularly, the disclosure provides methods and compositions for treating/regenerating mineralized tissue damaged by pulpal infection. In one embodiment, the tissue is selected from the group consisting of cementum, dentin, fibers (e.g., sharpy's fibers), periodontal ligament and bone. In one embodiment, the composition comprises a biocompatible material comprising (e.g., impregnated with) a PDGF protein (e.g., PDGF-BB and the like).

In some embodiments, the PDGF is present in the composition from about 0.01 mg/ml to about 10 mg/ml. The PDGF may be present in at any concentration within the range above. In other embodiments, PDGF is present at or between any one of the following concentrations: about about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.0 mg/ml; about 2.0 mg/ml, about 3.0 mg/ml; about 4.0 mg/ml, about 5.0 mg/ml; about 6.0 mg/ml; about 7.0 mg/ml; about 8.0 mg/ml; about 9.0 mg/ml; or about 10 mg/ml. Amounts of PDGF that can be used range from about 1 ug to about 50 mg, about 10 ug to about 25 mg, about 100 ug to about 10 mg, and about 250 ug to about 5 mg.

The PDGF (e.g., PDGF-BB) may be natural or recombinantly produced. As mentioned above PDGF-B can dimerize to form PDGF-BB. When produced by recombinant methods, a polynucleotide sequence encoding a monomer (e.g., PDGF B), can be engineered into a suitable vector and inserted into a prokaryotic or eukaryotic host cells for expression to subsequently produce the homodimer (e.g. PDGF-BB). As mentioned above commercially available GMP recombinant PDGF-BB can be obtained commercially from, e.g., Chiron Corporation (Emeryville, Calif.). Other suitable sources including R&D Systems, Inc. (Minneapolis, Minn.), BD Biosciences (San Jose, Calif.), and Chemicon, International (Temecula, Calif.). The nucleic acid sequences of PDGF-A, PDGF-B, and PDGF-C are known and the proteins have been cloned and expressed in various systems (see, e.g., HumanKine®, Proteintech; see also UniProt accession number P01127-1 PDGFB_HUMAN, which is incorporated herein by reference for all purposes).

The disclosure provides compositions and methods for treating and promoting regeneration of mineralized tissue in 1) apical periodontal chronic odontogenic infection and 2) chronic bony cystic defect (intabony, infrabony, or lateral defect) in a subject, the method comprising contacting the periapical or apical site with a composition comprising or consisting of a biocompatible material and an effective amount of platelet-derived growth factor (PDGF). In one embodiment, the biocompatible material comprises PDGF. In another embodiment, the PDGF is adsorbed or absorbed by a biocompatible material. In another embodiment, the PDGF is a recombinant PDGF. In still a further embodiment, the recombinant PDGF is recombinant human PDGF (rhPDGF). In still another embodiment, the PDGF is a PDGF-BB dimer. In another embodiment, the biocompatible material is xenogeneic to the subject to be treated. In another embodiment, the biocompatible material is allogeneic to the subject to be treated. In still another embodiment, the biocompatible material is synthetic.

By “periodontium” is meant the tissues that surround and support the teeth. The periodontium supports, protects and provides attachment and nourishment to the teeth. The periodontium consists of bone, cementum, periodontal ligament, and gingiva. Cementum is a thin, calcified layer of tissue that completely covers the dentin of the tooth root. Cementum is formed during the development of the root and throughout the life of the tooth and functions as an area of attachment for the periodontal ligament fibers.

The biocompatible material can be fibrous collagen material (e.g., a sponge), gelatin hydrogels, fibrin gels, heparin, reverse phase polymers such as the poloxamers, carriers composed of poly-lactic acid (PLA), poly-glycolic acid (PGA) or their co-polymers (PLGA), heparin-conjugated PLGA carriers, and inorganic materials such as calcium phosphates and/or beta tricalcium phosphate (R-TCP).

In one embodiment, the biocompatible material serves as a scaffold or a matrix for cell attachment. The shape of the biocompatible material can be any suitable shape for the tissue bed, however, in certain embodiments, the shape can be cylindrical or mostly conical in shape for easier filling into a root canal.

In certain embodiments, the biocompatible material comprises collagen, synthetic proteoglycans, gelatin, hydrogel, fibrin, phosphophorin, heparan sulfate, heparin, laminin, fibronectin, alginic acid, hyaluronic acid, chitin, PLA (polylactic acid), PLGA (lactic acid/glycolic acid copolymers), PEG (polyethylene glycol), PGA (polyglycol acid), PDLLA (poly-DL-lactic acid), PCL (polycaprolactone), chiason, hydroxyapatite, beta-TCP, calcium carbonate, titanium and gold. The proteoglycans above are composite sugars consisting of proteins and sugar chains (glucosaminoglycans) covalent bound to each other. The biocompatible material can be a sponge-shaped three-dimensional structure made of a collagen fiber having average diameter of 1 nm to 1000 nm.

“Biocompatible” refers to compounds or compositions and their corresponding degradation products that are relatively non-toxic and are not clinically contraindicated for administration into a tissue or organ.

The biocompatible material can take the form of a gel, matrix, film, or scaffold (e.g., a porous or non-porous material that can adsorb or absorb a growth factor or other active agents.

The biocompatible material may be of any material and/or shape that: (a) allows growth factors and optionally one or more additional active agents to adsorb or absorb thereto; and (b) allows cells to grow on or in the material. A number of different materials may be used to form the material, including but not limited to: polyglycolic acid (PGA), collagen (in the form of sponges, braids, or woven threads, etc.), cat gut sutures, cellulose, gelatin, or other naturally occurring biodegradable materials or synthetic materials, including, for example, a variety of polyhydroxyalkanoates. Any of these materials may be woven into a mesh, for example, to form a framework or scaffold.

The biocompatible material can be a naturally occurring alloplastic, xenogeneic or allogeneic bone preparation. For example, suitable allogeneic bone graft material or scaffolds are available under the trade names: Coreograft™ (beta-tricalcium phosphate), Corlok™, Duet™, Profuse™, Solo™, VG1® ALIF, VG2™ PLIF, VG2™ Ramp, Vertigraft VG2™ TLIF, Graftech™ products, Grafton™ products, Cornerstone-SR™, Cornerstone™ Select, MD™ Series, Precision™, Tangent™, Puros™, Vitoss™, Cortoss™, or Healos™. In certain embodiments, the biocompatible xenogeneic or allogeneic bone graft material can be in the form of a mesh, a gauze, a sponge, a monophasic plug, a biphasic plug, a paste, or a putty. In some embodiments, the biocompatible material can further comprise extracellular material materils such as Type I collagen, a Type II collagen, a Type III collagen, bovine collagen, human collagen, porcine collagen, equine collagen, avian collagen, or combinations thereof.

In some instances the biocompatible material is formed into a shape suitable for implantation into a site to be treated. For example, the biocompatible material can be formed into a plug to be inserted into a tissue socket. The plug or other design can be adsorbed with or absorbed with a PDGF protein (e.g., a PDGF-BB protein) prior to implantation, during or after implantation.

In some or further embodiments, the injury is the result of an infection, physical injury such as an accident or violence, or surgery (e.g., dental surgery).

In yet another embodiment, the disclosure provides a method of promoting growth and/or regeneration of periapical and/or or apical periodontal tissue, chronic intrabony pathology apical to the teeth roots and interradicular bony pathology at a site of injury, infection and/or trauma, wherein the method comprises applying a biocompatible material comprising or consisting of PDGF to the site or injury, or site to be repaired. In certain embodiments, the biocompatible material comprising or consisting of PDGF may further comprise additional factors to promote tissue growth and/or regeneration and/or to control infection. In certain embodiments, the site to be treated is first prepared to provide a suitable tissue bed for implantation of the biocompatible material. In some embodiments, the applied material contacts a tooth or teeth to promote tissue generation around the tooth or teeth.

As mentioned above, the biocompatible material comprising the PDGF can be allogeneic, xenogeneic or synthetic cell-free material. In addition, the biocompatible material can be in the form of a paste, plug, sponge etc. and can be formed to fit the site of repair.

As used herein reference to “a site” or “the site”, or “sites” refer to site in a subject that is to be treated and/or that has an infection, injury or damage. In some embodiments, the site of tissue to be repaired or regenerated is a mineralized tissue such as an apical, periodontal, periradicular, interradicular and/or periapical tissue. In another embodiment the site is a site of a root canal procedure.

An injury that can be treated by the methods and compositions of the disclosure includes injuries resulting from physical trauma. Such physical trauma can result from invasive medical procedures due to reconstructive surgery, periodontal surgery, jaw fracture and the like. Such surgeries may be due to infections from trauma, fungal, foreign body, virus or bacterial agents of the mouth and teeth.

The disclosure further demonstrates that apicoectomy+MTA showed partial reformation and regeneration of new cementum. Histological analyses confirmed that newly formed cementum did not have direct contact with MTA via collagen fibres. Partial healing of the periapical lesion reflected in a high bone volume loss. The outcome of this therapeutic modality was classified as repair.

MTA was the only retrograde material used in this study, since it had the most documented success rates compared to other retrograde filling materials. It has been recognized as an excellent root-end filling material for apical surgery due to its biocompatibility, antimicrobial efficacy, ability to set in a wet or bleeding environment, good sealing ability, and potential to promote biomineralization. It has also been reported that cementum can form over MTA via collagen fibril insertion. However, the results could not confirm the presence of functional fiber attachment on MTA or of any direct contact between the newly formed cementum and MTA.

When rhPDGF was added to the apicoectomy+MTA group, samples showed regeneration of cementum, periodontal ligament (PDL), and bone. Newly formed cementum covered the MTA completely in all cases, however gaps between the NFC and MTA were filled with fibrous-like tissue.

The disclosure also demonstrates that curettage in combination with rhPDGF yielded similar results to those of apicoectomy+MTA+rhPDGF as far as regeneration of PDL, cementum, and bone. Interestingly, radicular dentin regeneration occurred in this group and the newly formed cementum attached to radicular dentin had no gaps. This was the only group showing this functional attachment. The periapical tissue subjacent to the newly formed cementum, PDL, and bone was invariably free of all signs of inflammation when rhPDGF was used in addition to curettage or MTA. These findings suggest that rhPDGF has the potential to promote formation of a periapical tissue when applied onto a surgical wound following curettage or apicectomy and MTA retrograde filling. The newly formed tissue, after use of rhPDGF, showed no ankylosis. This supports the notion that rhPDGF can have the potential of stimulating cementogensis and dentogenesis in root development via cell activation.

As used herein, the term “subject” comprises a mammal. Exemplary mammals include: primates, such as monkeys, apes, and humans; pigs, cows, and other livestock; domesticated pets, such as dogs and cats; and other animals, such as horses. Typically, the subject is a human.

In certain embodiments, the disclosed methods can further comprise delivering or adding additional PDGF to a biocompatible material after implantation at the apical or periapical site. The additional PDGF can be delivered or added 1, 2, 3, 4, 5, 6, 7 days after implantation and/or application. In certain embodiments, the additional PDGF can be delivered or added once per week or several times per week after implantation. In some embodiments, a clinician can delivery or add additional PDGF to the biocompatible bone matrix material at suitable intervals and for a duration depending upon how regeneration or repair at the site of implantation is proceeding.

A biocompatible material used in the disclosure may be of any shape to provide proper bone formation. In some embodiments, pores or spaces in the material can be adjusted by one of skill in the art to allow or prevent migration of cells into or through the matrix material once implanted.

The invention has been generally described above and is further exemplified by the following examples, which are intended to illustrate but not limit the invention.

### EXAMPLES

Preoperative management. Six male beagle dogs were used for the study. The mean age and weight of the animals was 12±0.4 months and 13±1.2 kg, respectively. During the housing period, all subjects underwent supragingival scaling once a month using an ultrasonic scaler (NSK, Westborough, Mass.). Intramuscular (IM) antibiotics (25 mg/kg body weight) (Betamox LA. Norbrook Laboratory Limited. Newry. County Down, Northern Ireland) were administered one day prior to the procedure and followed by a second dose of the same antibiotic at the time of surgery. The dogs were anesthetized by intraperitoneal injection of xylazine (6-9 mg/kg, Lloyd Laboratories, Shenandoah, Iowa, USA) and ketamine (60-80 mg/kg, MilliporeSigma, St. Louis, Mo., USA).

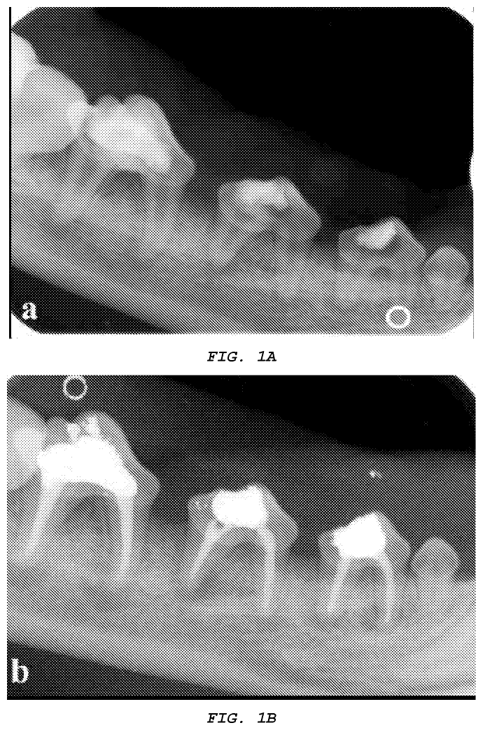

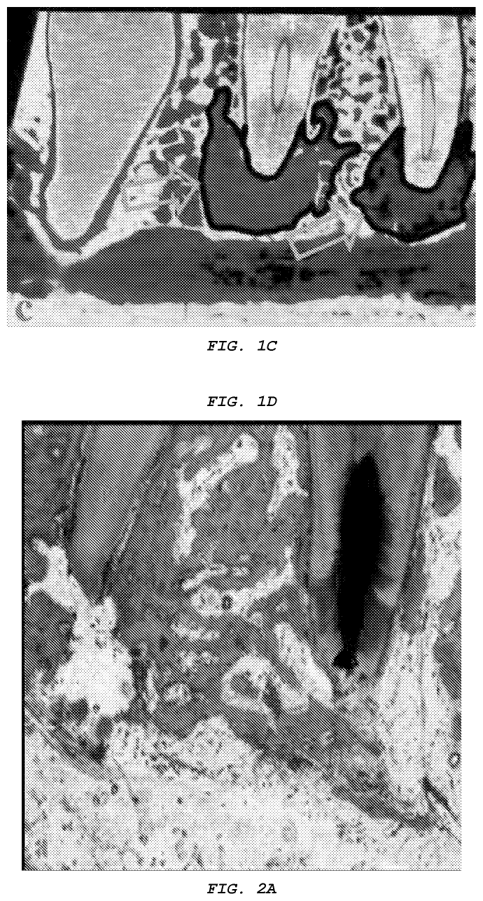

Induction of apical lesion. Premolars teeth were selected, and coronal access cavity performed in each tooth with a #2 size round tungsten bur (Brassler, Savannah, Ga.) mounted on a high-speed hand piece (Dentsply, York, USA). Sterile saline was used as coolant. Canal patency was verified by passing a #10 size K-file (Dentsply-Maillefer, Ballaigues, Switzerland) in the root canal until its tip extended 1 mm beyond the root apex as confirmed by Root ZX apex locator (Morita, Calif., USA). Following pulp extirpation using #15 size H-file (Dentsply-Maillefer, Ballaigues, Switzerland) the coronal access was left open for one week. At this point the canals were irrigated with saline and the coronal access was sealed with cotton pellet and IRM. Eight weeks later, periapical radiographs were taken and confirmed well-defined periapical radiolucent areas (FIG. 1A).

Animal grouping. The animals were randomly divided into four experimental groups by picking a paper from a brown bag labelled either “Group-1”, “Group-2”, “Group-3” or “Group-4”. Animal grouping was based on the accepted treatments of none healed chronic apical lesions following none-surgical root canal treatment (NSRCT). A total of sixty-four experimental teeth were distributed as follows: (Group 1) Apical curettage only; (Group 2) Apicoectomy+Mineral trioxide aggregate (MTA) root-end filling; (Group 3) Apicoectomy+MTA root-end filling+rhPDGF; and (Group 4) Apical curettage+rhPDGF. Additional 15 teeth were used as controls.

Root canal preparation. The root canals were instrumented using #10, 15 and 20 stainless steel K files (Dentsply-Maillefer, Ballaigues, Switzerland) to the working length, established 1 mm short of the apical foramen using Root ZX apex locator. Canal shaping was done using ProTaper rotary nickel-titanium files (Dentsply-Maillefer, Tulsa, Okla., USA) to F2 size (8% taper, 20/100 tip diameter) and a commercial preparation containing ethylenediaminetetraacetic acid (Glyde, Dentsply-Maillefer, Ballaigues, Switzerland). Canals were irrigated between each instrument with 2 ml 5.25% sodium hypochlorite, dried and obturated using warm vertical condensation of F2 calibrated gutta-percha points (Dentsply-Maillefer, Tulsa, Okla., USA), Obtura II (Obtura Spartan Endodontics, IL, USA) for backfill, and AH26 sealer (Dentsply—DeTrey, Konstanz, Germany). Subsequently the access cavities were sealed with amalgam.

Surgical protocol. Six months following completion of the NSRCT, the subjects were draped, pre-op periapical radiographs taken (FIG. 1B), and the surgical sites swabbed with an antiseptic solution (The Purdue Fredrick Company, Stamford, Conn.). Local anesthesia (Astra, Westborough, Mass.) was administered and a full thickness mucoperiosteal flap reflected to the mucco-gingival junction (MGJ) extending from mesial side of the canine to the mesial side of first molar using a #15 blade. Access to each lesion was done using Piezosurgery unit (Mectron, Piezosurgery® Columbus, Ohio, USA) and all granulation tissue removed. In group 1, a 1.5 mm root resection was done using a Piezo tip. In group 2, MTA was used as an apical plug following apicoectomy. In group 3, the apicoectomy+MTA was followed by application of rhPDGF (GEM21, Osteohealth, NY, USA) to fill the crypt. In group 4 apical curettage+rhPDGF. Retrograde cavity preparation was done by 1.5 mm root-end resection, followed by 3 mm depth retrograde cavity were performed under surgical magnification (×4), using p5 ultrasonic tip (Spartan, Mo., USA), Kis tips (KiS tips Spartan, Mo., USA) and water-cooling. In groups 3 and 4, GEM21 was applied to the root surface and the entire crypt space. In all cases, two suturing technique were used as follows: 1) Periosteal suture to the lingual flap using chromic gut 5-0 sutures (Universal sutures, Bangalore, India); and 2) Passive buccal flap closure with vertical mattress sutur using Vicril 5-0 (Ethicon, Johnson & Johnson Medical N.V., Belgium).

Postoperative management. All animals received antibiotics IM injections (25 mg/kg body weight, TID/day) (Betamox LA, Norbrook Laboratory Limited, Newry County Down, Northern Ireland) for 5 days. IM Analgesics (0.01-0.02 mg/kg, TID/day) (Buprenorphine, Idaho Falls, Id., USA) were administered immediately after surgery and for two days after surgery. Ten days after surgery, the mucosa was irrigated with sterile saline and the sutures removed.

Euthanasia. At the 32 month time period, all subjects were sacrificed using an intravenous overdose of 3% sodium pentobarbital (WA Butler Company, Dublin, Ohio, USA).

Hard tissue sectioning and histologic analyses. To remove the jaw segments containing all premolars and associated mesial and distal tooth structures en block, an electric saw was used (Leica SP 1600, Bannockburn, Ill., USA) and the jaws were fixed in 10% neutral formalin solution.

Micro computed tomographic (μCT) analysis. After fixation with 10% phosphate-buffered formaldehyde (pH 7.4) and dehydration in 70% ethanol, in vivo μCT was used to evaluate healing of the periradicular lesion. All subjects were assessed three-dimensionally using in vivo μCT (Sky Scan 1173, Brussels, Belgium) and scanned at 65 kV/385 mA source voltage/current, with a 1 mm aluminum filter. The pixel size (resolution), rotation step, and exposure time were 35 μm, 0.6° over 360, and 400 ms, respectively. The dataset was reconstructed with a software program (NRecon software, SkyScan, Belgium). Moderate beam hardening was applied in the reconstruction process. The μCT analysis was done with a CTAN software (SkyScan, Belgium). The Hounsfield Unit (HU) and bone mineral density (BMD) calibrations were first applied to the dataset. Two phantoms of calcium density 0.25 g/mm3 and 0.75 g/mm³ of 2/4 mm diameter (Gloor Instrument Switzerland) were scanned in a 10 ml Falcon tube filled with purified water using the scanning parameters described previously. Manufacturer's HU and BMD calibration procedures were then followed. The region of interest (ROI) was chosen as a 0.5 mm of thickness sleeve around each root apex individually (FIG. 1c; arrows). 3D images of the defect area were constructed using Insta Recon Software (EnterpriseWorks, 60 Hazelwood Dr., Champaign, Ill., USA). 30% Beam hardening effect reduction and 12% ring artifact correction were used to produce the precise image cross section.

After image reconstruction, two-dimensional virtual slices from the apical region of each tooth were acquired in the axial plane and examined corono-apically and mesio-distally to determine the first and last slices of which regenerative hard tissues could be identified. This provided a rough estimate of the extent of an unresolved periapical lesion, if present. Bone volume loss (BVL) in the area of induced chronic periapical lesion and region of interest (ROI) was defined and calculated (FIG. 1C) followed by calculation of BVL through 3D reconstructions of the ROI sections for each sample measured in cubic millimetres. To determine the normal space volume between the root apex and alveolar bone for a normal healthy tooth, measurements of 16 pristine teeth were performed in the same ROI of the treated group. The space around the apex of a normal canine premolar tooth was 1.51±3.55 mm³.

Light Microscopy. Jaw segments were decalcified for 10 weeks using a solution containing equal parts of 50% formic acid and 20% sodium citrate. Following decalcification, the specimens were washed in running water, dehydrated in an ascending ethanol series and embedded in paraffin. Polymerized blocks were primarily ground to bring the tissue components closer to the cutting surface. A section of 100 micrometer (μm) thickness attached to the second slide was cut using diamond blade saw under a pressure of 50 g to 100 g. An ultimate thickness of 40 μm was achieved by grinding and polishing each specimen with 1200, 2400, and 4000-grit sandpaper. Sections were stained with Toluidine blue/pyronin.

Histological analyses were performed using an image analysis system (OmniMet 9.5, Buehler, Lake Bluff, Ill., USA) linked to a light microscope. Pixel calibration was performed by using a digitized image of a stage micrometer for transmitted light (Ted Pella Inc, Redding, Calif., USA). Magnifications were between ×2 and ×32.

Results analysis. To assess the outcome of apical regeneration, histological analyses recorded the presence and pattern of the apical bone, periodontal ligament and apical cementum. The histological images were correlated with the corresponding μCT images. Outcome from both histological and μCT examinations was either regeneration or repair. Regeneration was defined as restoration of all lost structures (bone-PDL-cementum) and that the newly formed structures were similar in form and shape to the original ones. Repair was defined as partial restoration of the lost structures.

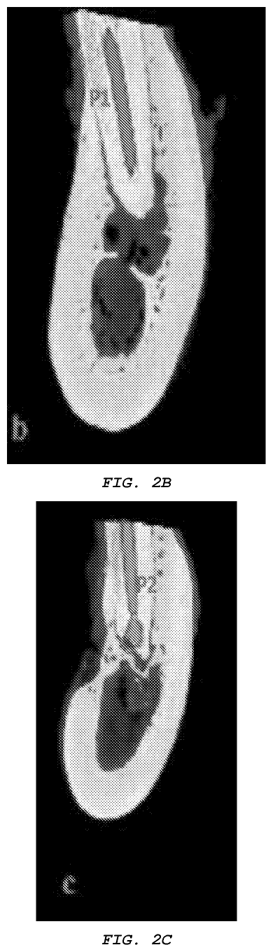

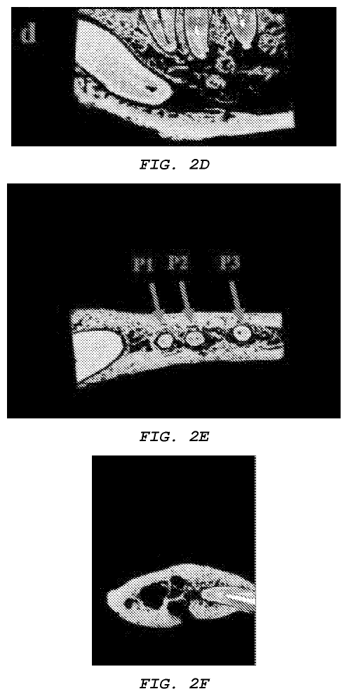

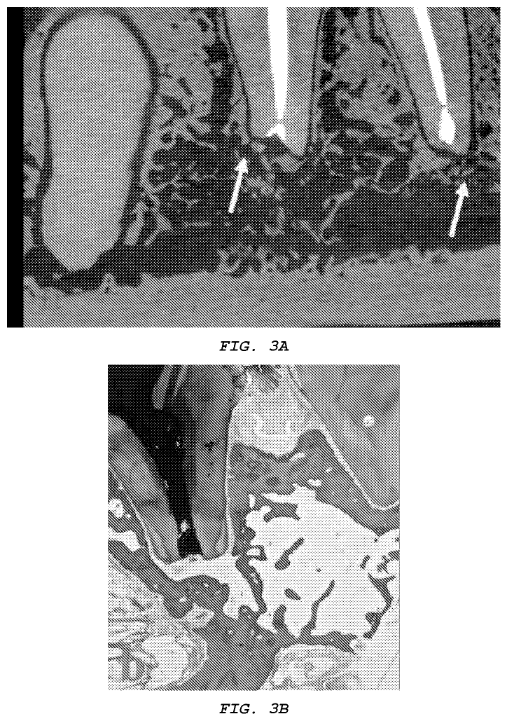

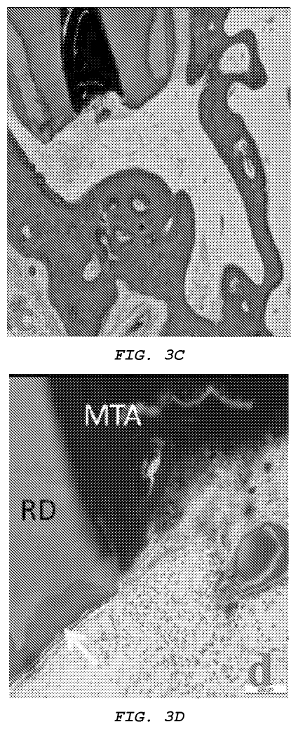

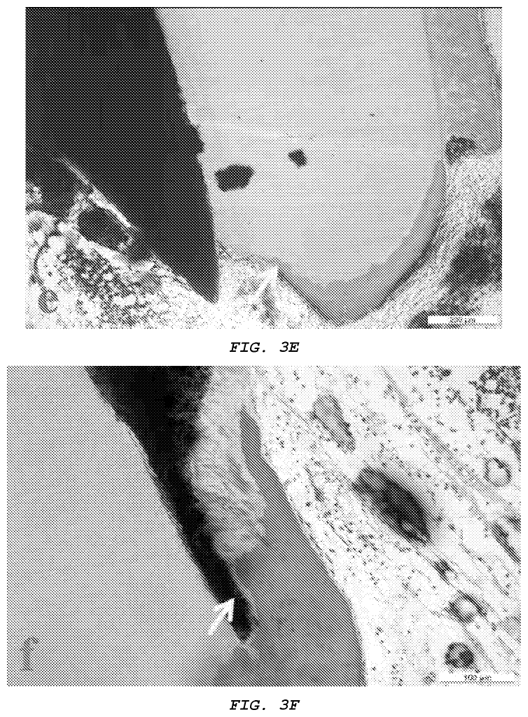

In group 1, histological analyses showed no evidence of bone-cementum-PDL regeneration (FIG. 2A; P1). Reconstructed axial μCT images of the periapical region showed large periapical lesion, absence of lamina dura and bone resorption (FIG. 2B; P1). The chronic periapical lesion borders were at proximity to the mandibular canal (FIG. 2A; P1 and FIG. 2C; P2) and extending 3 mm coronally (FIG. 2F). BVL in the apical area for the curettage alone group was 49.09±10.97 mm³. Curettage healing outcomes were classified as repair. In group 2, only nine teeth had newly-formed cementum (NFC) reformation over the apices (FIG. 2A; P2). Incomplete apical alveolar bone regeneration was evident in 11 specimens associated with incomplete cementum formation over the apex (FIG. 3A-C). NFC has a direct contact with the existing old cementum (FIG. 3E) but no direct contact with MTA by means of collagen fibril attachment (FIG. 2A; P2; FIG. 3D-F). Fibrous-like tissues can be seen between the NFC and MTA (FIG. 3G, H). A corresponding reconstructed μCT images (FIG. 2C; P2, and FIG. 3a) show partial resolution of the periapical lesion. BVL for the apicoectomy+MTA group was 35.34±10.97 mm³. Repair was the outcomes for this type of therapeutic approach.

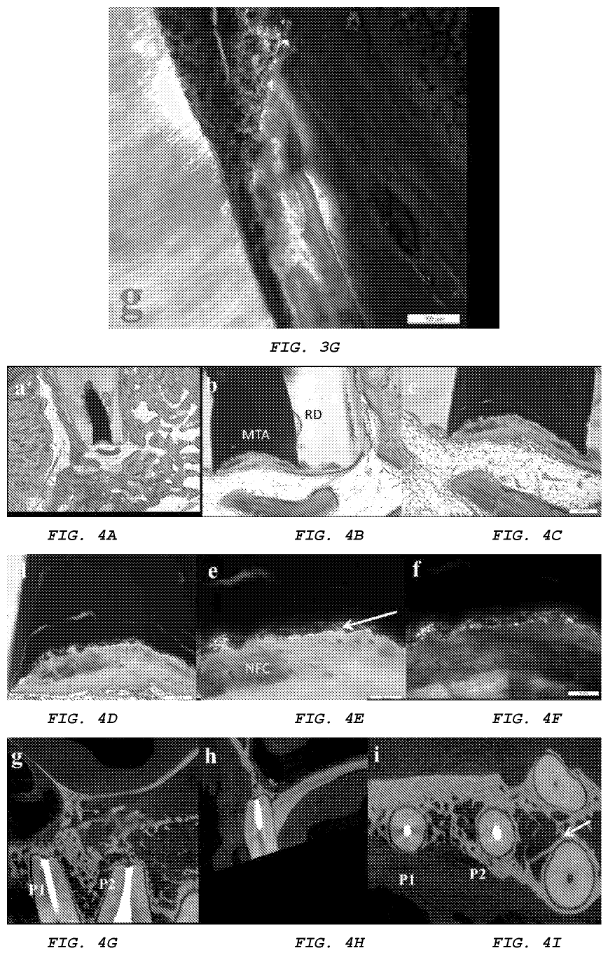

In group 3 (apicoectomy+MTA+rhPDGF), evidence of regenerated periodontal ligament, bone and apical cementum in 14 of the 16 specimens was seen (FIG. 4a-c). There was no direct contact between the NFC and MTA and the gaps were filled with fibrous-like tissues (FIG. 4C-F). μCT images and 3D analyses shows complete resolution of the periapical lesions (FIG. 4G-I). BVL loss for this group was 4.51±1.55 mm3. The outcomes for this therapeutic approach was classified as regeneration.

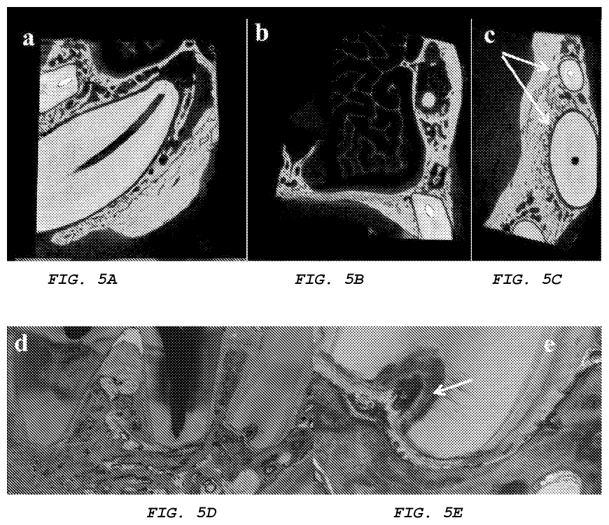

In group 4 (curettage+rhPDGF), regeneration of cementum, periodontal ligament and apical bone was seen in all the specimens (FIG. 5C-D). The newly formed cemental tissues were characterized by even thickness, absence defects, and by direct functional attachment to radicular dentin. Only this group showed evidence of radicular dentin regeneration (FIG. 5D). μCT analyses show regeneration of apical bone with consistent width of the lamina dura despite in its proximity to maxillary sinus in some cases (FIG. 5A). BVL for this group was 2.82±2.3 mm³. Outcomes of this group was classified as regeneration.

A number of embodiments of the invention have been described. Nevertheless, it will be understood that various modifications may be made without departing from the spirit and scope of the invention. Accordingly, other embodiments are within the scope of the following claims.

What is claimed is:

1. A method of promoting periapical tissue healing and regeneration, the method comprising contacting an apical or periapical site with a composition consisting of recombinant Platelet Derived Growth Factor (rPDGF).

2. The method of claim 1, wherein the rPDGF is recombinant human PDGF (rhPDGF).

3. The method of claim 1, wherein the composition containing the rPDGF is selected from the group consisting of collagen, gelatin hydrogels, fibrin gels, heparin, reverse phase polymers, poloxamers, poly-lactic acid (PLA), poly-glycolic acid (PGA), co-polymers of PLA and PGA (PLGA), heparin-conjugated PLGA carriers, and inorganic materials.

4. The method of claim 3, wherein the inorganic material is calcium phosphates and/or beta tricalcium phosphate (R-TCP).

5. The method of claim 3, wherein the collagen materials are selected from Type I collagen, Type II collagen, Type III collagen, bovine collagen, human collagen, porcine collagen, equine collagen, avian collagen, and any combination thereof.

6. The method of claim 1, further comprising administering antibiotics, antifungals or a combination thereof.

7. The method of claim 1, wherein the apical or periapical site is a site of injury.

8. The method of claim 7, wherein the injury is the result of a periodontal infection or surgery.

9. The method of claim 1, wherein the composition contacts a tooth or teeth.
